(19) United States
(12) Patent Application Publication
(10) Pub. No.: US 2008/0095869 A1
(43) Pub. Date: Apr. 24, 2008

(54) Anticancer Treatment

(76) Inventors: David Archibald, West Perth (AU); Dorothy Morre, West Lafayette, IN (US); D. James Morre, West Lafayette, IN (US)

Correspondence Address: NATH & ASSOCIATES, 112 South West Street, Alexandria, VA, 22314, US

(21) Appl. No.: 11/660,277

(22) PCT Filed: Aug. 17, 2005
(86) PCT No.: PCT/AU05/01245

§ 371 Date: Jan. 4, 2008

(30) Foreign Application Priority Data
Aug. 19, 2004 (AU) 2004904700

(51) Int. Cl. A61K 36/00; A61K 31/165; A61P 35/00

(52) U.S. Cl. 424/760; 514/622

(57) ABSTRACT

A method of inhibiting tNOX in a living entity which includes administering to the entity, wherein the entity has cancer cells that express tNOX, a therapeutically active amount of a combination of botanicals selected from the groups consisting of cruciferous vegetables and Capsicum plants.

### FIELD OF THE INVENTION

The present invention relates to compositions and method of treatment providing improved inhibition of tNOX.

In particular, the present invention relates to compositions and method of treatments that selectively inhibit tNOX and thus inhibit the growth of cancerous cells.

### DESCRIPTION OF THE PRIOR ART

Cancer is a cellular phenomenon of uncontrolled growth. Normal cells in a mature animal divide in a controlled manner. Cancer-specific cells arise by abnormal and unregulated growth, which can eventually destroy surrounding body tissue. In many instances, cancer may also spread to other parts of the body in a process called metastasis.

In 2003 US mortality statistics, cancer is responsible for 23% of all deaths, with the risk to men of developing cancer being 1 in 2 and women 1 in 3.

Modern therapies in the treatment of cancer involve the use either chemotherapy or radiation therapy or surgery in an attempt to remove the cancer.

Chemotherapy involves the use of various complex drugs, many of which are synthesized in a laboratory. Such drugs are often given in combination with other compounds with the aim of disrupting the growth cycle of the cancer cells.

Many of these drugs have significant side effects on humans including hair loss, vomiting, nausea, and reduced white blood cell count, which can lead to an increased possibility of the patient receiving a secondary infection. This is due to the fact that many existing drug cancer treatments affect normal cells. The presence of these detrimental side effects can lead to lack of efficacy due to lack of patient compliance with the drug-taking regime.

Additionally, synthetic drugs are often difficult to produce, requiring significant investments in both time and financial resources that are ultimately passed on the patient. The availability and cost of cancer treatments is an important factor in the patient deciding to proceed with a specific chemotherapy.

A major extant problem of human health is the need for inexpensive, safe and effective methods of cancer prevention and treatment (Cooper, Elements of Human Cancer, Jones and Bartlett, Boston, 1992).

The use of natural plant extracts in treating different diseases in known. By way of example only, JP 10-236968 discloses the use of extracts of paradicsom paprika to inhibit cancer cells in a concentration-dependent manner. However, in order to achieve this it is first necessary to extract the appropriate compounds from the plant using organic solvents such as acetone and hexane.

In U.S. Pat. No. 5,830,887 there is a disclosure of a method for treating cancer using compositions enriched with natural phyto-oestrogens or analogues thereof that are selected from Genistein, Daidzein, Formononetin and Biochanin A. Such phyto-oestrogens are available from soya hypocotyl and red clover.

There is a strong trend towards the use of naturally derived compounds for the effective treatment of cancer. Patients are more likely to be accepting of a drug that they know has been derived from natural sources such as plants as mere is the belief that the side effects of such drugs will not be severe as those that would be considered unnatural.

Additionally, when the main source of the active component is available from natural sources this can then lead to the final cancer therapy or drug to be significantly lower in price, and thus more available.

Compounds derived from Capsicum plants have been used as an anaesthetic (U.S. Pat. No. 4,313,958 and U.S. Pat. No. 4,493,848). Capsicum compounds have also been combined with other analgesic compounds, such as non-steroidal anti-inflammatory drugs (NSAID) (U.S. Pat. No. 4,812,446) or opioids (U.S. Pat. No. 4,599,342). The combination of these different classes of analgesic compounds produced synergistic effects, in that the combination produced greater analgesic effects than either compound alone.

U.S. Pat. No. 5,665,378 describe a transdermal therapeutic composition, administered in patch form, comprising capsaicin, NSAID and pamabrom. The NSAIDs used include diflunisal, fenoprofen, ibuprofen, indomethacin, meclofenamate, naproxen etc.

Capsicum-based compounds have also been used in other compositions for treating ailments such as arthritis, strains, bruises and sprains on the outside of the patient, mainly in patch form but there are also a number of creams an aerosols for topical application.

In most instances, along with the active Capsicum compound, there is, by necessity, a second ingredient that in some way reduces the skin irritation caused by the capsaicin. Usually this is a skin anaesthetic or a compound, which binds to the capsaicin.

From the above, the main focus of compositions and methods of treatment involving Capsicum-based compounds has been relieving the effects of topical diseases.

Cruciferous vegetables, such as cauliflower, cabbage, and kale contain sulforaphane, which is an isothiocyanate that is a known antioxidant. Sulforaphane and other isothiocyanates are believed to be responsible for the lowered risk of cancer that is associated with the consumption of broccoli and other cruciferous vegetables.

However, the use of such isothiocyanates is merely seen as being mildly prophylactic.

### OBJECT OF THE INVENTION

It is an object of the present invention to provide a method of treating cancer.

More specifically, it is an object of the present invention to provide a synergistic combination, composition and a method of treatment of living entitles each of which is useful in providing in a different way than hitherto inhibition of tNOX.

More specifically, it is an object of the present invention to provide for a method of treating cancer by increasing the activity of sulforaphane.

Another object of the invention is to overcome, or at least substantially ameliorate, the disadvantages and shortcomings of the prior art.

Other objects of the invention and advantages of the present invention will become apparent from the following description, taken in connection with the accompanying figures, wherein, by way of illustration and example, an embodiment of the present invention is disclosed.

### SUMMARY OF THE INVENTION

According to the present invention, which following statement is not intended to be necessarily the only or indeed the broadest form of this, there is provided a method of inhibiting tNOX in a living entity which includes administering to the entity, wherein the entity has cancer cells that express tNOX, a therapeutically active amount of a combination of botanicals selected from the groups consisting of cruciferous vegetables and Capsicum plants.

In preference the cruciferous vegetable is broccoli.

In preference, said Capsicum plants are derived from the Capsicum annum species.

In preference, finely powdered fruits of the Capsicum plants are used.

In preference, said fruits contain Capsicum vanilloids.

In preference, said vanilloids are capsaicin and/or vanillylamine.

In preference, said broccoli contains sulforaphane.

In a further form of the invention there is provided a method of treating cancer in a patient in need of cancer therapy comprising administering to said patient by ingestion an anticancer effective amount of a composition including a product of at least two plants selected from the group of Capsicum plants and cruciferous vegetables.

In preference, the product of the Capsicum plant is finely powdered dried fruit.

In preference, the cruciferous vegetable is broccoli.

In preference, the product of the broccoli is selected from the group of finely ground broccoli sprouts, commercially available broccoli sprouts, and a solution of broccoli sprout extract.

In preference, the solution of broccoli sprout extract is an aqueous extract.

In preference, the method of treating cancer involves introducing into the mammal in combination at least the two said extracts to an extent that they are active to provide synergistic activation and at least over time there will be effected by these materials in combination an inhibition of tNOX activity of the cancer cell.

In preference, said anticancer effective amount by weight of dried extract of broccoli as compared to the dried Capsicum annum fruits is between 10:1 and 100:1.

In preference, said broccoli is broccoli sprouts.

In preference, the broccoli sprouts are lyophilised.

In preference, the composition includes a pharmaceutically acceptable carrier.

In yet a further form of the invention there is disclosed a method of treating cancer in a patient in need of cancer therapy comprising administering to said patient by ingestion an anti-cancer effective amount of a combination of a purified capsaicinoid and sulforaphane in a physiologically acceptable formulation.

In preference, the capsaicinoid is derived from the powdered fruits of a Capsicum annum cultivar and/or its constituents.

In preference, the sulforaphane originates from lyophilised broccoli sprouts.

The term “pharmaceutically acceptable carrier” is intended to mean, but not limited to, a non-toxic solid, semisolid or liquid filler, diluents, encapsulating material or formulation auxiliary of any type.

In a further form, the invention can be said to reside in a method of treatment of a living entity to inhibit replication of cancer cells within that entity where the entity is of a type that has a life-sustaining process and where a tumour will express tNOX uniquely in contradiction to any expression from normal or non-cancer cells, the method including the steps of introducing into the entity so as to be effectively active within the entity over at least a substantial time together, therapeutic materials which are an extract of a en cruciferous vegetable (including a substantial quantity of sulforaphane) and Capsicum or an extract of Capsicum, in which there is a synergistic effect that leads to the improvement in the effect of the cruciferous vegetable extract.

In preference the extract of Capsicum is a vanilloid-containing Capsicum preparation.

In a further form the invention can be said to reside in a therapeutic material for the treatment of tumours in living entities which material (whether as a mixture or cooperatively packaged or administered or sold together) is 100 units by weight of broccoli extract and from 1-10 units by weight of Capsicum extract.

In preference, said Capsicum extracts are derived from the Capsicum annum species.

In preference, said Capsicum extract are finely powdered fruits of the Capsicum plant.

In preference, said Capsicum extracts contain Capsicum vanilloids.

In preference, said vanilloids are capsaicin and/or vanillylamine.

In preference, said broccoli extract contains sulforaphane.

In a further form of the invention this can be said to reside in a botanical supplement consisting of lyophilised broccoli sprouts combined with powdered chillies (Capsicum annum species) in ratios of weight between 10:1 and 100:1 whereby tumour cell division inhibitory activities of the broccoli sprouts on both the tNOX and cell culture assays are enhanced synergistically.

A unique plasma membrane NADH oxidase (NOX), a unique cell surface protein with hydroquinone (NADH) oxidase and protein disulfide-thiol interchange activities that is responsive to hormone and growth factors has been identified. Further, a hormone-insensitive and drug-responsive form of NOX designated tNOX, which is specific to cancer cells has been reported.

Because the NOX protein is located at the external plasma membrane surface and is not transmembrane, a functional role as an NADH oxidase is not considered likely. While the oxidation of NADH provides a basis for a convenient method to assay the activity, the ultimate electron physiological donor is most probably hydroquinones with specific activities for hydroquinone oxidation greater than or equal to that of NADH oxidation and/or protein thiol-disulfide interchange.

CNOX was originally defined as a drug-indifferent constitutive NADH oxidase activity associated with the plasma membrane of non-transformed cells that was the normal counterpart to tNOX. Indeed, a 36 kD protein isolated from rat liver and from plants has NOX activity that is unresponsive to tNOX inhibitors.

While cancer cells exhibit both drug-responsive and hormone and growth factor-indifferent (tNOX) as well as drug inhibited and hormone and growth factor dependent (CNOX) activities, non-transformed cells exhibit only the drug-indifferent, hormone- and drug-responsive CNOX. Among the first descriptions of so-called constitutive or CNOX activity of non-transformed cells and tissues was where the activity of rat liver plasma membranes was stimulated by the growth factor, diferric transferrin. Subsequent work demonstrated that the observed NADH oxidation was catalysed by a unique enzyme exhibiting responsiveness to several hormones and growth factors. Unlike mitochondrial oxidases, the hormone-stimulated NADH oxidase activity of rat liver plasma membranes is not inhibited by cyanide. The enzyme also was distinguished from other oxidase activities by its response to several common oxidoreductase inhibitors, e.g., catalase, azide and chloroquine, as well as to various detergents e.g., sodium cholate, Triton X-100 and CHAPS. Like tNOX of cancer cells, CNOX is a unique membrane-associated protein that is capable of oxidizing NADH but has an activity which is modulated by hormones and growth factors.

There remains a need for treatment of cancer that does not have the adverse effects generally caused by the non-selective of conventional chemotherapeutic agents.

Inhibition of tNOX, an extracellular membrane-associated protein, by the above-mentioned combination of Capsicum and broccoli products, results in the selective inhibition of cancer cell growth and ultimately, apoptosis.

What is now provided is a way or method of treating cancer that was until now unknown. The method enhances the activity, to a previously unknown level, of sulforaphane, a major anticancer ingredient of broccoli, by combination with Capsicum vanilloids such as capsaicin and vanillylamine. Both the sulforaphane and the Capsicum vanilloids target the cancer-associated and growth-related ECTO-NOX protein tNOX, Efficacy evaluations are based on inhibition of tNOX activity of human cervical carcinoma (HeLa) cells and of growth of HeLa and 4T1 (mouse mammary carcinoma) cells in culture. Synergy of inhibition is observed for sulforaphane and the vanilloids in both systems. Specifically, a claim is made for a novel botanical supplement consisting of lyophilised broccoli sprouts combined with powdered chillies (Capsicum annum species) in ratios between 10:1 and 100:1 where activities of the broccoli sprouts on both the tNOX and cell culture assays are enhanced 2- to 5-fold by the combination compared to broccoli sprouts or chilli powders alone when compared at the same relative concentrations.

### BRIEF DESCRIPTION OF THE DRAWINGS

FIG. 1. Inhibition of NOX activity (fully oxidized) from the HeLa cell surface by sulforaphane.

FIG. 2. Inhibition of NOX activity (no H202) from the HeLa cell surface by sulforaphane.

FIG. 3. Inhibition of NOX activity (fully oxidized) of 4T1 mouse mammary cells by sulforaphane.

FIG. 4. Sulforaphane does not inhibit NOX activity of human mammary (non-cancer) epithelia which lack tNOX.

FIG. 5. Sulforaphane does not inhibit NOX activity of plasma membranes isolated from dark-grown hypocotyls of soybean which lack tNOX.

FIG. 6. Effect of sulforaphane on growth of HeLa and human mammary carcinoma (BT-20) cells in culture at 48 and 72 h of treatment.

FIG. 7. A-D. Effect of sequential additions of sulforaphane, capsaicin and EGCg on NOX activity from the HeLa cell surface added in the order given from left to right.

FIG. 8. Effect of sequential additions of sulforaphane, vanillylamine and EGCg on NOX activity from the HeLa cell surface added in the order given from left to right.

FIG. 9. Inhibition of NOX activity from the HeLa cell surface by broccoli extract alone (A) and in combination with various pepper powders.

FIG. 10. Survival of HeLa and 4T1 cells after 72 h of treatment with different dilutions of broccoli extract alone.

FIG. 11. Survival of HeLa (A and C) and 4T1 (B and D) cells comparing two different sources of pepper (Capsicum annum) powder; A, B, Ancho; C, D. Piquin. The optimum ratio for combination is one part pepper powder to 25 parts lyophilised broccoli sprouts.

FIG. 12. As in FIG. 11 except a mixture of two pepper powders. The optimum ratio again is 1 part pepper powder to 25 parts lyophilised broccoli sprouts.

FIG. 13. NADH oxidase activity comparing different ratios of lyophilised broccoli sprouts and pepper powders. The optimum ratio for inhibition was 1 part pepper powder to 25 parts lyophilised broccoli sprouts.

FIG. 14. Survival of LnCap (human prostate cancer) cells in culture and response to extract of lyophilised broccoli sprouts (BSL) with and without pepper powder (PP) in a 25:1 ratio. G=guiallijo. A=ancho.

### DETAILED DESCRIPTION OF THE INVENTION

This invention has as its basis the discovery of a cell surface NADH oxidase activity with utility as a screening method for potential anticancer agents.

Among the more potent NOX inhibitors are capsaicin (8-methyl-N-vanillyl-6-noneamide), the pungent principle of chilli peppers and EGCg((−)-epigallocatechin gallate), the principal tea catechin. In this application, we describe compositions consisting of powdered fruits of Capsicum annum cultivars and/or its constituents plus lyophilised broccoli sprouts and/or their constituents with potential utility in the treatment and/or prevention of cancer.

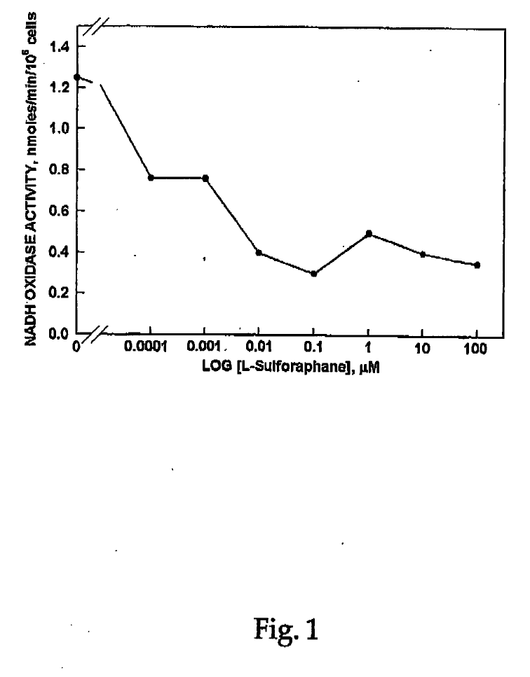

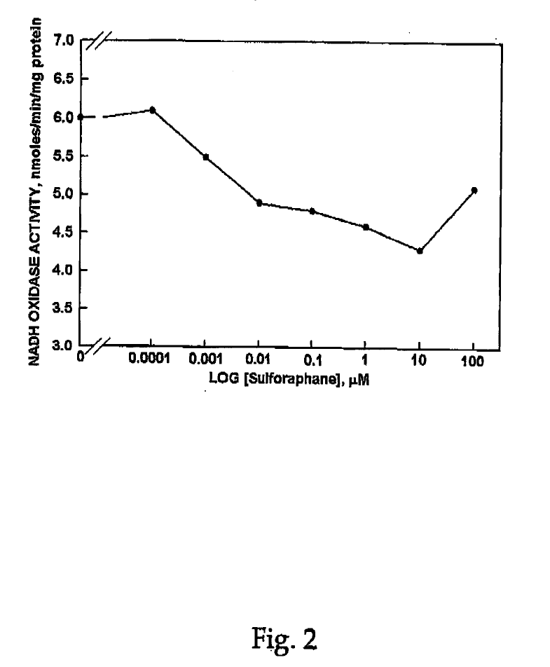

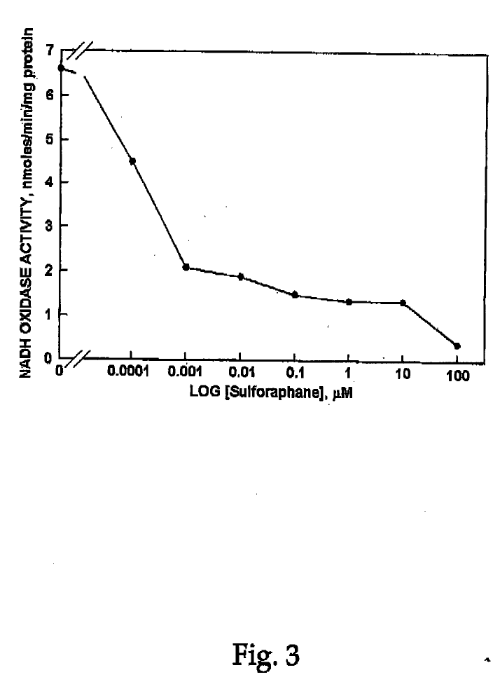

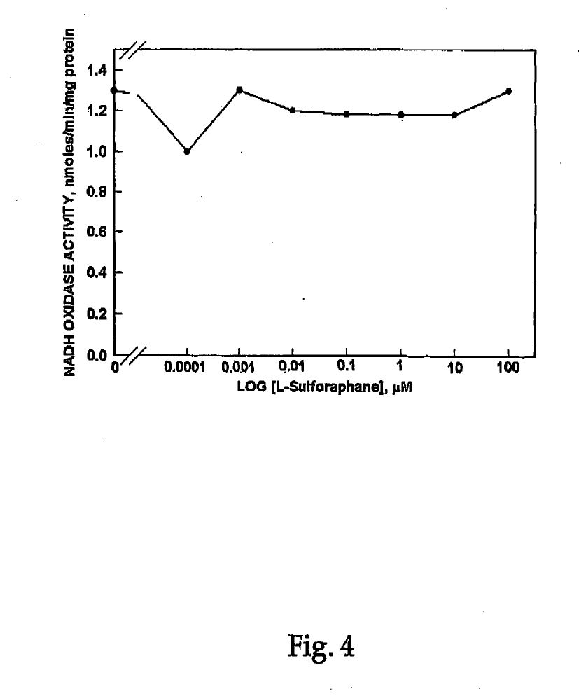

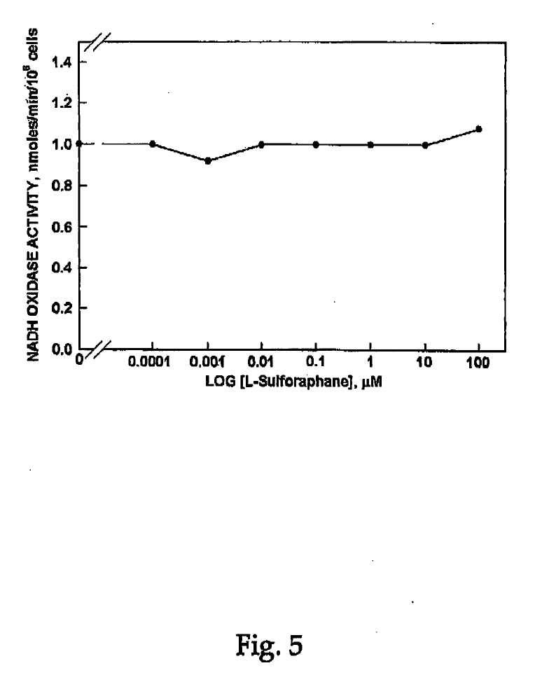

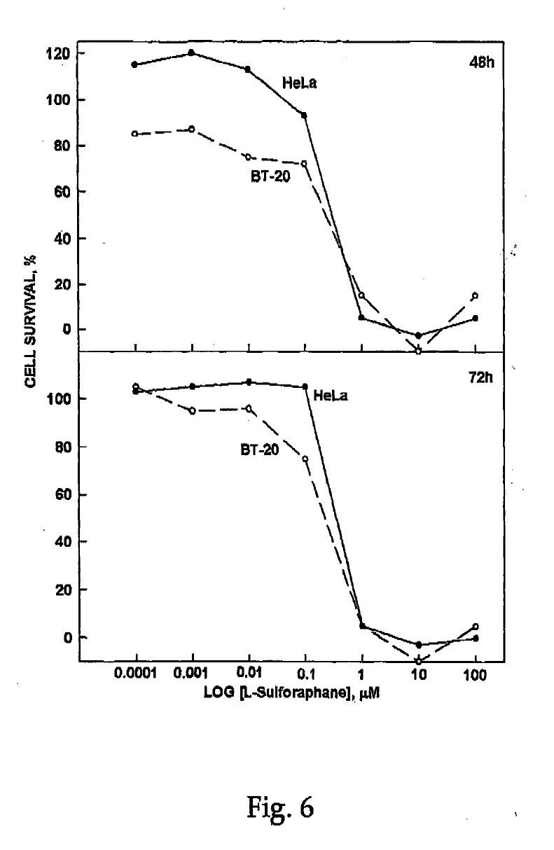

L-Sulforaphane (sulforaphane), an isothiocyanate prevalent in broccoli that blocks initiation of cancer caused by chemicals, was shown to be a potent inhibitor of the tNOX cancer target. Activity for L-Sulforaphane with an EC₅₀ of about 1 mM was shown for tNOX from HeLa (FIGS. 1 and 2) and for tNOX of 4T1 mouse mammary cells (FIG. 3). CNOX of non-cancer MCF-10A human mammary epithelia was unaffected by L-Sulforaphane (FIG. 4) as was the CNOX activity of soybean plasma membranes (FIG. 5). L-Sulforaphane inhibited the growth of HeLa and human mammary cancer (BT-20) cells with an EC₅₀ of between 0.1 and 1 mM (FIG. 6). The margin of safety, however, with growth of cells was less than a factor of 10 with non-cancer MCF-10A cells being inhibited to nearly the same extent as the cancer cells.

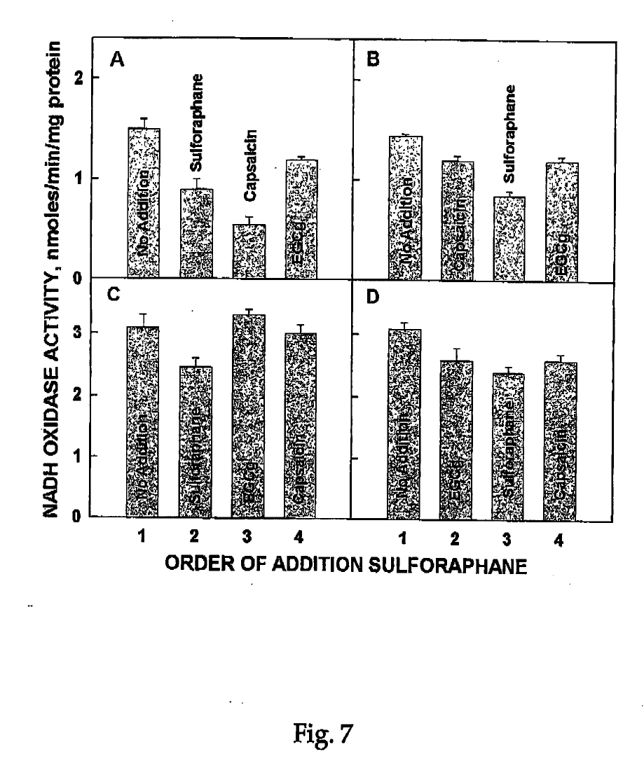

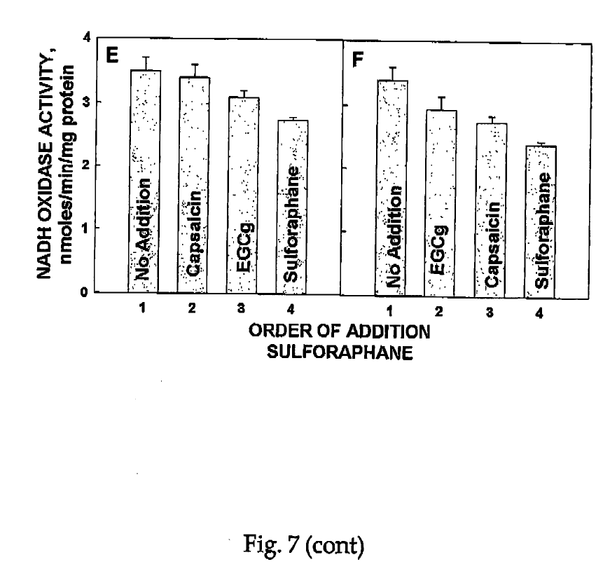

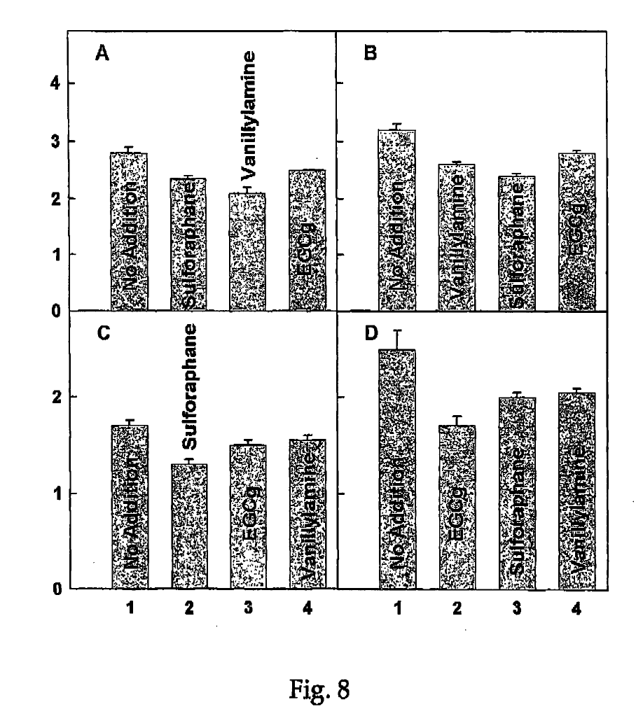

When sulforaphane was combined with other tNOX inhibitors from natural sources, capsaicin, vanillylamine and EGCg, a beneficial response was seen with capsaicin (FIG. 7) and with vanillylamine (FIG. 8) either preceding (FIG. 7B, FIG. 8B) or following (FIG. 7A, FIG. BA) the addition of sulforaphane, EGCg was antagonistic in all combinations (FIG. 7C-F, FIG. 8C-F) as was green tea.

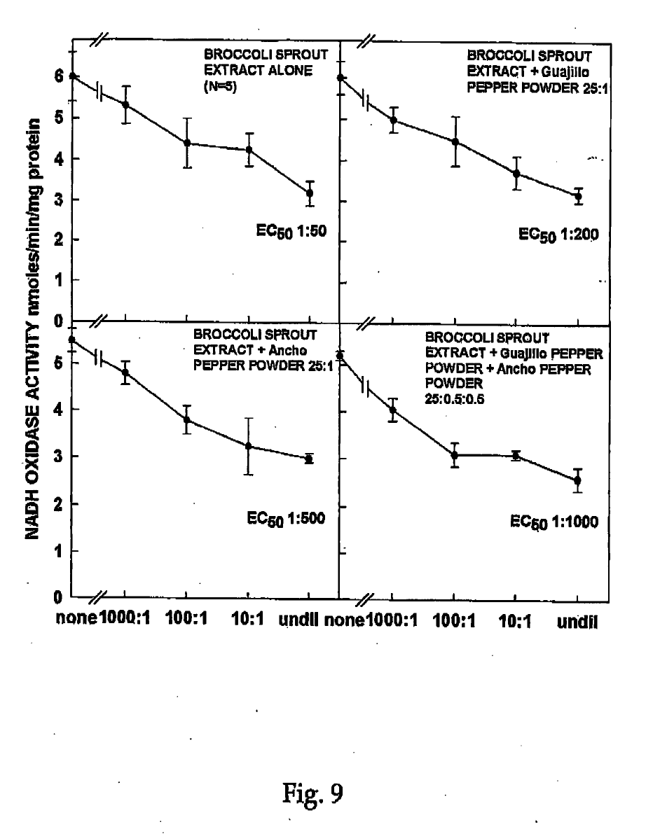

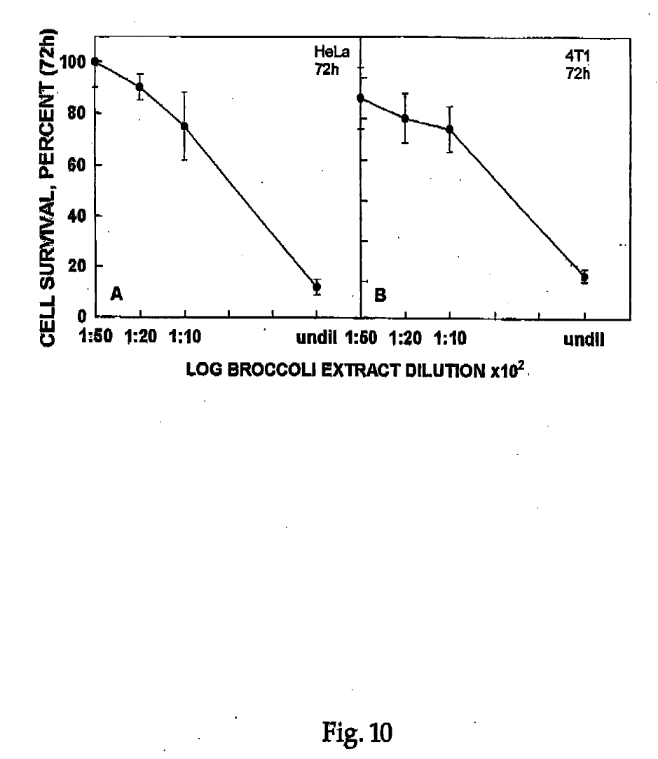

For development of a botanical based on these observations, lyophilised broccoli sprouts were used. Dark grown seedlings from organic broccoli seeds (Sun Organic Farm, San Marcos, Calif.) germinated at 25° C. and 4 days old were harvested, frozen and lyophilised directly. Finely ground and sieved sprouts or a commercial preparation of sprouts (Arizona Health Foods) were cold water extracted overnight prior to assay (FIG. 9). Standard broccoli extracts 1.25 mg/ml were diluted 1:10, 1:20 or 1:50 and added to HeLa or 4T1 cells grown in a 96 well format at a further dilution of 1:100. The EC₅₀ for inhibition of growth of HeLa and 4T1 cells by the broccoli extract was 1:500 (final dilution) (FIG. 10).

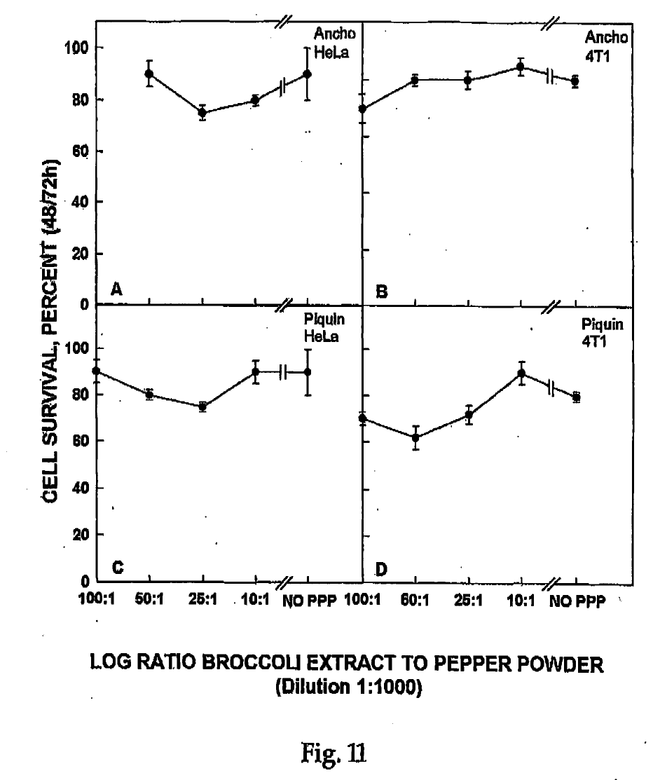

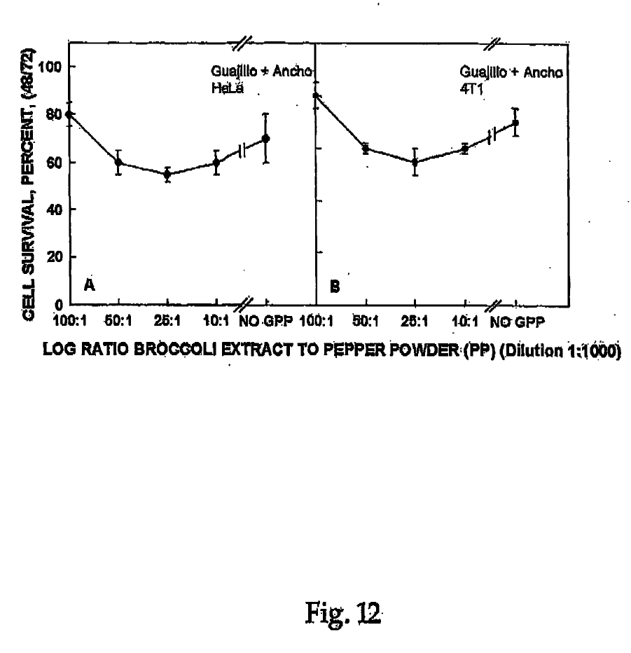

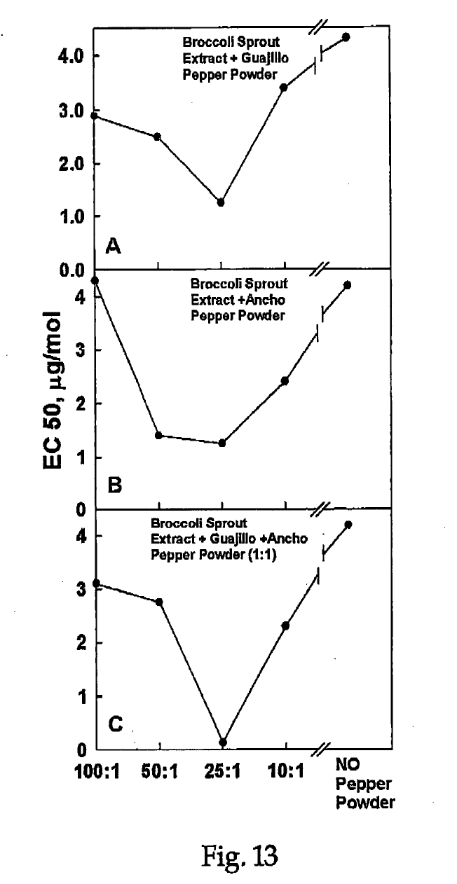

Combination of broccoli extract with finely powdered Capsicum annum fruits of different varieties (pepper powders) enhanced the cell killing of the broccoli preparation by a factor of approximately 5 (FIG. 11). Powders mixed from two different peppers were more active than single sources (FIG. 12). The optimum ratio for combination of lyophilised broccoli sprouts and pepper powder was determined to be 25 parts lyophilised broccoli sprouts to 1 part pepper power (FIG. 12). Similar results were obtained for inhibition of NADH oxidase activity from the HeLa cell surface (FIG. 13).

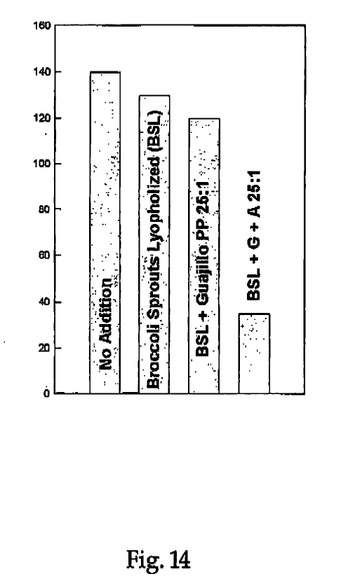

Growth of LnCap (human prostate cancer) cells in culture was inhibited by lyophilized broccoli sprout extract but required addition of a mixture of pepper powders in the 25:1 ratio to achieve a strong inhibitory response (FIG. 14).

Growth of Cells

HeLa (ATCC CCL-2) human cervical adenocarcinoma cells were cultured in minimal essential medium (Eagle), with 2 mM L-glutamine and Earle's balanced salt solution adjusted to contain 1.5 g/L sodium bicarbonate, 0.2 mM non-essential amino adds, 1.0 mM sodium pyruvate and supplemented with 10% bovine calf serum (heat-inactivated) plus 50 mg/L gentamycin sulfate (Sigma).

The 4T1 mammary cancer cell line arose from a BALB/c C3H mouse (Miller et al., 1987). The 4T1 cells were grown in DME-10, Dulbecco's modified Eagle's medium supplemented with 5% foetal calf serum, 5% newborn calf serum, 1 mM mixed non-essential amino acids, 2 mM L-glutamine, penicillin (100 U mL⁻¹) and streptomycin (100 mg mL⁻¹).

Preparation of HeLa Cells and Cell-Free Extracts

HeLa S cells (grown in suspension) were collected by centrifugation and shipped frozen by a commercial supplier (Cellex Biosciences, Minneapolis, Minn.) In 0.1 m sodium acetate, pH 5, in a ratio of 1 mL packed cell volume to 1 mL acetate. The cells were thawed at room temperature, resuspended and incubated at 37° C. for 1 h to release the protein. The cells were removed by centrifugation at 37000 g for 60 min and the cell-free supernatants were refrozen and stored in 1 mL samples at −70° C.

Spectrophotometric Assay of NADH Oxidase

NADH oxidase activity was determined as the disappearance of NADH measured at 340 nm in a reaction mixture containing 25 mM Tris-Mes buffer (pH 7.2), 1 mM KCN to inhibit low levels of mitochondrial oxidase activity, and 150 (J-M NADH at 37° C. with temperature control (±0.5°) and stirring (14). Activity was measured using paired Hitachi U3210 spectrophotometers. Assays were initiated by addition of NADH. With plasma membranes and whole cells, assays were for 1 min and were repeated on the same sample every 1.5 min for the time indicated. A millimolar extinction coefficient of 6.22 was used to determine specific activity. Proteins were estimated by the bicinchoninic acid method with bovine serum albumin as standard.

Growth Measurements

Growth was determined using a 96-well plate assay as described by Lin et al. (17). HeLa (5×10⁴) or CHO (10⁴) cells were distributed into each plate well (Costar tissue culture plate). The cells were grown at 37° C. for 24 h after which the substances to be evaluated were added followed by incubation for an additional 48 or 72 h as indicated. Medium was removed and the cells were washed with phosphate-buffered saline and then fixed by addition of 100 mL 2.5% (v/v) glutaraldehyde for 0.5 h followed by a distilled water wash. The cells were stained with 100 nL 1% aqueous crystal violet for 0.5 h, washed exhaustively with distilled water followed by 200 mL 33% (v/v) acetic acid for 5 min. The absorbance was determined at 580 nm using an automated plate reader. Growth was determined according to the formula [(b−c)×100]/(a−c)] where a=absorbance of cells in medium without treatment b=cells in medium with treatment and c=medium alone (background).

From these results we have established that in sera results indicate significant synergistic effects resulting from use of the two indicated materials together and that these results will indicate equivalent beneficial enhancement in viva.

In Vivo Trial Results

Three patents with elevated PSA (prostate specific antigen) levels were provided with the combination of broccoli extract with finely powdered Capsicum annum fruits of different varieties (pepper powders) as described previously. Each patient ingested 700 mg of the combination of broccoli extract with finely powdered pepper powders every four hours.

In each case there was a marked response to the PSA levels, showing a reduction in the overall rate of increase of PSA levels or levelling out of PSA levels.

One patient, prior to treatment, had a PSA level that was increasing at 13.3% over an 8 month period. During treatment with the combination of broccoli extract with finely powdered pepper powders, the rise on PSA was reduced to only an increase of 2.78%.

Another patient had PSA levels that were recoded at rising by 5 units per day prior to the distraction of the combination of broccoli extract with finely powdered pepper powders. During the 14 days that the patient was ingesting the combination, their PSA levels leveled off and started to decline slightly. After completion of the trial, the patients PSA levels resumed rising at 6 units per day.

The purpose of this description is to illustrate the invention and not limit it.

Although the invention has been hearing shown and described in one is conceived to be the most practical and preferred embodiment, it is recognized that departures can be made within the scope of the invention, which is not to be limited to the details described herein but it is to be accorded the full scope of the appended claims so as to embrace any and all equivalent methods.

1-40. (canceled)

41. A method of inhibiting tNOX in a living entity having cancer cells that express tNOX, which includes administering to the entity a synergistic therapeutically effective amount of a combination of Capsicum plants and aqueous extract from cruciferous vegetables.

42. The method of claim 1, wherein the cruciferous vegetable is broccoli.

43. The method of claim 42, wherein the broccoli contains sulforaphane.

44. The method of claim 43, wherein the ration of broccoli extract to Capsicum plants is between 10:1 and 100:1 wiw,

45. The method of claim 44, wherein the ration of broccoli extract to Capsicum plants is between 25:1

46. The method of claim 45, wherein the Capsicum plants are derived from the Capsicum annum species.

47. The method of claim 46, wherein finely powdered fruits of the Capsicum plants are used

48. The method of claim 47, wherein the fruits contain Capsicum vanilloids.

49. The method of claim 48, wherein the vanilloids are selected from the group consisting of capsaicin and vanillylamine.

50. The method of claim 49, wherein the broccoli contains sulforaphane.

51. A method of treating cancer in a patient in need of cancer therapy comprising administering to said patient an synergistic anti-cancer effective amount of a composition including a product of at least a plant selected from the group of Capsicum plants and an aqueous extract of cruciferous vegetables.

52. The method of claim 51, wherein product of the Capsicum plant is finely powdered dried fruit.

53. The method of claim 52, wherein the fruits contain Capsicum vanilloids.

54. The method of claim 53, wherein the vanilloids are selected from the group consisting of capSafoin and vanillylamine.

55. The method of claim 54, wherein the cruciferous vegetable is broccoli.

56. The method of claim 55, wherein the product of the broccoli is selected from the group of finely ground broccoli sprouts, commercially available broccoli sprouts, and a solution of broccoli sprout extract.

57. The method of claim 56, wherein the solution of broccoli sprout extract is an aqueous extract.

58. The method of claim 57, wherein the method of treating cancer involves introducing into the mammal in combination at least the two said extracts to an extent that they are active to provide synergistic activation and at least over time there will be effected by these materials iri combination an inhibition of tNOX activity of the cancer cell.

59. The method of claim 58, wherein the anti-cancer effective amount by weight of extract of broccoli as compared to the dried Capsicum annum fruits is between 10:1 and 100:1.

60. The method of claim 59, wherein the anti-cancer effective amount by weight of extract of broccoli as compared to the dried Capsicum annum fruits is 25:1.

61. The method of claim 60, wherein the broccoli extract is broccoli sprouts.

62. The method of claim 61, wherein the broccoli sprouts are lyophilised.

63. The method of claim 62, wherein the composition includes a pharmaceutically acceptable carrier.

64. A method of treating cancer in a patient in need of cancer therapy comprising administering to the patient an synergistic anti-cancer effective amount of a combination of a purified capsaicinoid and sulforaphane in a physiologically acceptable formulation.

65. The method of claim 61, wherein the capsaicinoid is derived from the powdered fruits of a Capsicum annum cultivar and/or its constituents.

66. The method of claim 62, wherein the sulforaphane originates from lyophilised broccoli sprouts.

67. A composition for treating cancer in a living entity, wherein the cancer is a type having cancer cells that express tNOX, wherein the composition includes a synergistic therapeutically active amount of a combination of Capsicum plants and an aqueous extract of cruciferous vegetables.

68. The composition of claim 67, wherein the cruciferous vegetable is broccoli.

69. The composition of claim 68, wherein the Capsicum plants are derived from the Capsicum annum species.

70. The composition of claim 69, wherein the fruits of the Capsicum plants are finely powdered.

71. The composition of claim 70, wherein the fruits contain Capsicum vanilloids.

72. The composition of claim 71, wherein the vanilloids are selected from the group consisting of capsaicin, vanillylamine.

73. The composition of claim 72, wherein the broccoli contains sulforaphane.

74. The composition of claim 73, wherein the broccoli is lyophilised broccoli sprouts.

75. The composition of claim 74, wherein the lyophilised broccoli sprouts combined with powdered chillies (Capsicum annum species) in ratios of weight between 10:1 and 100:1

76. The composition of claim 75, wherein the lyophilised broccoli sprouts combined with powdered chillies (Capsicum annum species) in ratios of weight 25:1.

77. A method of inhibiting the division of cancer cells in a living entity, wherein the cancer is a type having cancer cells that express tNOX, the method including administering to the living entity a synergistic combination of Capsicum plants and an aqueous extract of cruciferous vegetables so as to provide a prophylactic treatment of cancer by inhibiting the expression of tNOX within the cancer cells.

78. The method of claim 77, wherein the cruciferous vegetable is broccoli.

79. The method of claim 78, wherein the broccoli contains sulforaphane.

80. The method of claim 79, wherein the Capsicum plants are derived from the Capsicum annum species.

81. The method of claim 80, wherein finely powdered fruits of the Capsicum plants are used

82. The method of claim 81, wherein the fruits contain Capsicum vanilloids.

83. The method of claim 82, wherein the vanilloids are selected from the group consisting of capsaicin and vanillylamine.

84. A composition for treating cancer in a living entity, wherein the cancer is a type having cancer cells that express tNOX, wherein the composition includes a synergistic therapeutically active amount of a combination of capsaicin and sulforaphane.

85. The composition of claim 84, wherein the ratio of capsaicin to sulforaphane is between 10:1 and 100:1 w/w.

86. The composition of claim 85, wherein the capsaicin is sourced from Capsicum plants.

87. The composition of claim 86, wherein the sulforaphane is sourced from cruciferous vegetables.
